(19) United States
(12) Patent Application Publication
(10) Pub. No.: US 2013/0112220 A1
(43) Pub. Date: May 9, 2013

(54) PROCESS FOR MAKING UP OR CARING FOR KERATIN FIBRES USING RETRACTABLE FIBRES, AND USE THEREOF

(71) Applicant: Guillaume Kergosien, Chaville (FR)

(72) Inventor: Guillaume Kergosien, Chaville (FR)

(73) Assignee: L'OREAL, Paris (FR)

(21) Appl. No.: 13/704,376

(22) PCT Filed: Jun. 10, 2011
(86) PCT No.: PCT/IB2011/052545

§ 371 Date: Jan. 24, 2013

Related U.S. Application Data
(60) Provisional application No. 61/370,988, filed on Aug. 5, 2010.

(30) Foreign Application Priority Data
Jun. 16, 2010 (FR) 1054769

(51) Int. Cl. A61K 8/85; A61Q 1/00; A45D 40/26

(52) U.S. Cl. CPC A61K 8/85; A45D 40/26; A61Q 1/00
USPC 132/206; 132/216; 424/70.11

(57) ABSTRACT

Composition for making up and/or caring for keratin fibres, comprising fibres that are capable of changing in length and/or shape in response to an external stimulus.

The present invention relates to the field of making up and caring for keratin fibres, especially the eyelashes, and more particularly concerns mascara compositions.

Eyelash coating compositions such as mascaras are generally makeup compositions, cosmetic eyelash care compositions or compositions to be applied over a makeup, also known as top coats.

Mascaras are generally classified according to two types: water-based mascaras, also known as “cream mascaras”, which comprise a dispersion of wax(es) in water, and anhydrous mascaras with a low water content, also known as “waterproof mascaras”, which comprise a dispersion of wax(es) in one or more organic solvents.

The present invention more specifically relates to water-based mascaras. Besides the care and/or makeup effect afforded by mascaras, their users also want mascaras to be able to curl the eyelashes.

Thus, standard mascaras can generally curl the eyelashes, but the degree or extent of curling obtained generally remains limited.

Devices also exist for increasing the level of curling of the eyelashes, for instance heating combs, which are used after the application of the mascara, or eyelash curling tongs, which exist in heating or non-heating version. However, the use of devices such as eyelash curling tongs is rather complex and does not really make it possible to obtain uniform curling of the eyelashes, these devices generally leading to folding of the eyelashes in places.

In general, devices for curling the eyelashes do not always lead to genuinely satisfactory results.

The problem posed by the present invention is that of proposing a composition and a process that can produce improved curling of the eyelashes, i.e. that can produce eyelashes whose curling is more pronounced and also produce uniform curling: i.e. all the treated eyelashes are also curled.

The inventors of the present patent application have, surprisingly and unexpectedly, solved this problem by means of a composition for making up and/or caring for keratin fibres, comprising, in a physiologically acceptable medium, fibres that are capable of changing in length and/or shape in response to an external stimulus.

The external stimuli for changing the length or shape are chosen in particular from:
the application of a source of heat or of cold,
the application of a liquid, especially water at room temperature, at a temperature above room temperature or at a temperature below room temperature,
the application of an electrical current especially in the form of a short electric discharge or in the form of a low-intensity current,
the application of radiation of a given wavelength, for example UV radiation,
the application of a source of vibration or friction.

Under the effect of the stimulus, the fibres change in state, in particular their dimension(s), i.e. their length and/or diameter may decrease or they may change in conformation and thus, for example, become curled.

A first subject of the present patent application is directed towards a composition for making up and/or caring for keratin fibres, in particular a mascara composition comprising a physiologically acceptable medium and containing fibres that are capable of changing in length and/or shape in response to an external stimulus, the said fibres being made of a material chosen from polyethylene terephthalate and derivatives thereof.

In particular, it is a composition in which the fibres are fibres whose length and/or volume decreases, i.e. fibres that are retractable at a temperature T₁, which is less than or equal to 80° C. The fibres used are then also referred to as “heat-retractable fibres”.

According to one preferred variant, the composition according to the invention is free of wax whose melting point is less than or equal to T₁.

A second subject of the present patent application is directed towards a process for making up and/or caring for keratin fibres according to the present invention, which comprises at least the following steps, in this order:
application of a composition comprising a physiologically acceptable medium and containing fibres that are retractable at a temperature T₁, which is less than or equal to 80° C., the said fibres being made of a material chosen from polyethylene terephthalate and derivatives thereof,
heating the keratin fibres to a temperature ranging from T₁ to 100° C.

Preferably, the temperature T₁ ranges from 60 to 80° C., and consequently the fibres used are retractable at a temperature ranging from 60 to 80° C.

A third subject of the present patent application is directed towards the use in a mascara composition of fibres that are capable of changing in length and/or shape when they are subjected to a temperature of less than or equal to 80° C., the said fibres being made of a material chosen from polyethylene terephthalate and derivatives thereof, for increasing the level of curling of the eyelashes and/or for increasing the uniformity of curling of the eyelashes.

The subject of the present invention leads to visibly greater curling of the eyelashes.

The rest of the description, especially the examples, will demonstrate other aspects, properties and advantages of the present invention.

Heat-Retractable Fibres

The fibres used in the composition used in the process according to the present invention are formed from one or more materials chosen from polymers with a temperature of change of state ranging from 45 to 70° C.

The term “fibre” should be understood as meaning an object of length L and of diameter D such that L is greater than D and preferably very much greater than D, D being the diameter of the circle in which the cross section of the fibre is inscribed. In particular, the ratio L/D (or aspect ratio) is chosen in the range from 3.5 to 2500, preferably from 5 to 500 and better still from 5 to 150.

For the purposes of the present patent application, the term “temperature of change of state of a material” will be understood as meaning its glass transition temperature or its melting point in the case of a semi-crystalline material. In the case of amorphous materials, the glass transition temperature is measured using a dynamic mechanical analyser (DMA), for example a dynamic mechanical analyser sold under the name DMA Q800 by the company TA Instruments. In the case of semi-crystalline materials, a differential scanning calorimeter (DSC), for example the calorimeter sold under the name DSC 30 by the company Mettler, is used for measuring the melting point of the crystalline chains.

Preferably, the temperature of change of state of the material(s) used for forming the heat-retractable fibres ranges from 45° C. to 65° C.

The material(s) used for forming the heat-retractable fibres are chosen from polyethylene terephthalate and derivatives thereof.

For the purposes of the invention, the term “polyethylene terephthalate and derivatives thereof” means polyethylene terephthalate homopolymers and copolymers, and also mixtures of polyethylene terephthalate (co)polymers with any other polymer(s).

Preferably, the polyethylene terephthalate and derivatives thereof are chosen from polyethylene terephthalate copolymers, polyethylene terephthalate glycol (PETG) copolymers available especially from the company Eastman Chemical, and mixtures of polybutylene terephthalate and polyethylene terephthalate.

Without wishing to be bound by any theory, it appears that a polymer with an excessively low temperature of change of state will retract prematurely at a temperature to which it would be brought under normal storage conditions, and that a polymer with an excessively high glass transition temperature will require an excessive heating temperature to obtain the retraction effect, which will run the risk of being uncomfortable, or even of injuring the user.

In a particularly preferred manner, the polymer used for forming the heat-retractable fibres is a copolymer of terephthalic acid, of ethylene glycol and of diethylene glycol such as PETG 5116 available from the company Eastman Chemical Products.

Without wishing to be bound by any theory, it appears that the fibres used in the compositions used according to the present invention are made heat-retractable by placing them in a dimensionally unstable state. This unstable state is obtained by modifying the orientation of the molecular chains generally by longitudinally or biaxially stretching the material at a temperature above the temperature of change of state and maintaining the stretching constraint during cooling.

Preferably, the heat-retractable fibres that may be used in the present patent application are such that their degree of retraction ranges from 25% to 85% and preferably from 40% to 70% after having been placed in a bath of water at 70° C.

The term “retraction at 70° C.” (TR₇₀) means:

TR₇₀=(size of the fibre before treatment−size of the fibre after treatment)/size of the fibre before treatment

The manufacture of heat-retractable filaments is known to those skilled in the art. It consists in stretching a polymer filament to a ratio ranging from 1/1.5 to 1/6 in a bath of water heated to a temperature ranging from 60° C. to 100° C.; such a process that is suitable for the manufacture of retractable filaments that may be used in the present invention is especially described in patent U.S. Pat. No. 4,108,845. It is then possible to chop the heat-retractable filaments obtained into small-sized fibres that may be used in the mascara compositions according to the present invention.

The heat-retractable fibres that may be used in the process according to the present invention may also be made by performing the process described in patent U.S. Pat. No. 5,439,438. This process consists in extruding a polymer for forming fibres through the orifices of an extruder die in a stream of gas at high speed so as to transform the polymer into fibres. The fibres obtained are then directed towards the entry of a chamber placed close to the die and positioned in a direction parallel to the path of the fibres when they leave the die. Air is blown into the chamber along the axis of the chamber at a sufficient speed to keep the fibres under tension during their passage through the chamber. The fibres are collected when they leave the opposite side of the chamber.

Preferably, the heat-retractable fibres that may be used in the process according to the present invention have a length ranging from 0.1 mm to 10 mm, preferably from 0.5 mm to 5 mm and better still from 1 mm to 3 mm.

Advantageously, the heat-retractable fibres that may be used in the process according to the present invention have a diameter ranging from 5 to 50 μm and preferably from 10 to 50 μm.

Advantageously, the amount of heat-retractable fibres ranges from 5% to 50% by weight and preferably from 10% to 20% by weight relative to the total weight of the mascara composition.

According to one preferred embodiment, the process for making up and/or caring for keratin fibres according to the present invention is performed using a mascara composition comprising at least one wax with a melting point of greater than 80° C. The melting point of the wax is measured using a differential scanning calorimeter (DSC), for example the calorimeter sold under the name DSC 30 by the company Mettler.

In particular, the waxes are chosen from hard waxes with a hardness of greater than 6 MPa. The hardness is determined by measuring the compression force, measured at 20° C. using the texturometer sold under the name TA-TX2i by the company Rheo, equipped with a stainless-steel cylindrical spindle 2 mm in diameter, travelling at a measuring speed of 0.1 mm/second, and penetrating the wax to a penetration depth of 0.3 mm. The hard wax is preferably chosen from polar hard waxes such as carnauba wax, candelilla wax and polyethylene waxes. Even more preferably, the hard wax is chosen from carnauba wax and candelilla wax.

Advantageously, the amount of wax(es) with a melting point of greater than 80° C. ranges from 0.1% to 50%, preferably from 1% to 25% and even more preferably from 5% to 15% by weight relative to the total weight of the mascara composition.

The wax(es) may be present in the form of an aqueous wax microdispersion. The term “aqueous wax microdispersion” means an aqueous dispersion of wax particles, in which the size, expressed as the volume-median effective diameter D[4.3], of the said wax particles is less than or equal to about 1 μm.

Wax microdispersions are stable dispersions of colloidal wax particles, and are described especially in Microemulsions Theory and Practice, L. M. Prince Ed., Academic Press (1977) pages 21-32.

In particular, these wax microdispersions may be obtained by melting the wax in the presence of a surfactant, and optionally of some of the water, followed by gradual addition of hot water with stirring. Intermediate formation of an emulsion of the water-in-oil type is observed, followed by a phase inversion with final production of a microemulsion of the oil-in-water type. On cooling, a stable microdispersion of solid colloidal wax particles is obtained.

The wax microdispersions may also be obtained by stirring the mixture of wax, surfactant and water using an agitation means such as ultrasonication, a high-pressure homogenizer or turbomixers.

The particles of the wax microdispersion preferably have mean sizes of less than 1 μm (especially ranging from 0.02 μm to 0.99 μm), preferably less than 0.5 μm (especially ranging from 0.06 μm to 0.5 μm).

These particles are formed essentially from a wax or a mixture of waxes. They may, however, comprise a minor proportion of oily and/or pasty fatty additives, a surfactant and/or a common additive/liposoluble active agent.

According to another preferred variant, the composition according to the invention is free of waxes.

The compositions according to the invention may also comprise any ingredient conventionally used in the field of mascaras, for instance pasty compounds, film-forming polymers, gelling agents, fillers and additional fibres.

Film-Forming Polymers

Among the film-forming polymers that may be used in the compositions of the present invention, mention may be made of synthetic polymers, of free-radical type or of polycondensate type, and polymers of natural origin, and mixtures thereof.

The expression “free-radical film-forming polymer” means a polymer obtained by polymerization of unsaturated and especially ethylenically unsaturated monomers, each monomer being capable of homopolymerizing (unlike polycondensates).

The film-forming polymers of free-radical type may especially be vinyl polymers or copolymers, especially acrylic polymers.

The vinyl film-forming polymers may result from the polymerization of ethylenically unsaturated monomers containing at least one acidic group and/or esters of these acidic monomers and/or amides of these acidic monomers.

Monomers bearing an acid group that may be used include α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), especially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl(meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates that may be mentioned are hydroxyethyl acrylate, 2-hydroxypropyl acrylate, hydroxyethyl methacrylate and 2-hydroxypropyl methacrylate.

Among the aryl(meth)acrylates that may be mentioned are benzyl acrylate and phenyl acrylate.

The (meth)acrylic acid esters that are particularly preferred are the alkyl (meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned previously.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene.

Among the film-forming polycondensates that may be mentioned are polyurethanes, polyesters, polyester-amides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea-polyurethanes, and mixtures thereof. An example that may especially be mentioned is the polyurethane sold under the commercial reference Baycusan C1001 by the company Bayer.

The polyesters may be obtained, in a known manner, by polycondensation of dicarboxylic acids with polyols, especially diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, mention may be made of: sulfoisophthalic acid, sulfoterephthalic acid, sulfophthalic acid and 4-sulfonaphthalene-2,7-dicarboxylic acid.

The copolymers preferably used are those based on isophthalate/sulfoisophthalate, and more particularly copolymers obtained by condensation of diethylene glycol, cyclohexanedimethanol, isophthalic acid and sulfoisophthalic acid.

The polymers of natural origin, optionally modified, may be chosen from shellac resin, sandarac gum, dammar resins, elemi gums, copal resins and cellulose-based polymers, and mixtures thereof.

According to a first embodiment of the invention, the film-forming polymer may be a water-soluble polymer and may then be present in the aqueous continuous phase of an emulsion.

According to another variant, the film-forming polymer may be a polymer dissolved in a liquid fatty phase comprising organic solvents or oils such as those described hereinbelow (the film-forming polymer is thus said to be a liposoluble polymer). Preferably, the liquid fatty phase comprises a volatile oil, optionally mixed with a non-volatile oil, the oils possibly being chosen from the oils mentioned below.

Examples of liposoluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Liposoluble film-forming polymers that may also be mentioned include liposoluble copolymers, and in particular those resulting from the copolymerization of vinyl esters containing from 9 to 22 carbon atoms or of alkyl acrylates or methacrylates, the alkyl radicals containing from 10 to 20 carbon atoms.

Such liposoluble copolymers may be chosen from copolymers of polyvinyl stearate, polyvinyl stearate crosslinked with divinylbenzene, with diallyl ether or with diallyl phthalate, polystearyl(meth)acrylate copolymers, polyvinyl laurate and polylauryl(meth)acrylate, these poly(meth)acrylates possibly being crosslinked with ethylene glycol dimethacrylate or tetraethylene glycol dimethacrylate.

The liposoluble copolymers defined above are known and described especially in patent application FR-A-2 232 303; they may have a weight-average molecular weight ranging from 2000 to 500 000 and preferably from 4000 to 200 000.

Liposoluble homopolymers may also be mentioned, and in particular those resulting from the homopolymerization of vinyl esters containing from 9 to 22 carbon atoms or of alkyl acrylates or methacrylates, the alkyl radicals containing from 2 to 24 carbon atoms.

Examples of liposoluble homopolymers that may especially be mentioned include: polyvinyl laurate and polylauryl(meth)acrylates, these poly(meth)acrylates possibly being crosslinked using ethylene glycol or tetraethylene glycol dimethacrylate.

According to one advantageous embodiment, a composition according to the invention comprises at least one polyvinyl laurate film-forming polymer.

As liposoluble film-forming polymers that may be used in the invention, mention may also be made of polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers that may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned include those sold by the company Wacker under the reference Resin MK, such as Belsil PMS MK, or by the company Shin-Etsu under the reference KR-220L.

Siloxysilicate resins that may be mentioned include trimethyl siloxysilicate (TMS) resins such as those sold under the reference SR 1000 by the company General

Electric or under the reference TMS 803 by the company Wacker. Mention may also be made of the trimethyl siloxysilicate resins sold in a solvent such as cyclomethicone, sold under the name KF-7312J by the company Shin-Etsu, and DC 749 and DC 593 by the company Dow Corning.

Mention may also be made of silicone resin copolymers such as those mentioned above with polydimethylsiloxanes, for instance the pressure-sensitive adhesive copolymers sold by the company Dow Corning under the reference Bio-PSA and described in document U.S. Pat. No. 5,162,410, or the silicone copolymers derived from the reaction of a silicone resin, such as those described above, and of a diorganosiloxane, as described in document WO 2004/073 626.

It is also possible to use silicone polyamides of the polyorganosiloxane type, such as those described in documents U.S. Pat. Nos. 5,874,069, 5,919,441, 6,051,216 and 5,981,680.

These silicone polymers may belong to the following two families:

polyorganosiloxanes comprising at least two groups capable of establishing hydrogen interactions, these two groups being located in the polymer chain, and/or

polyorganosiloxanes comprising at least two groups capable of establishing hydrogen interactions, these two groups being located on grafts or branches.

According to one embodiment of the invention, the film-forming polymer is a film-forming linear block ethylenic polymer, which preferably comprises at least one first block and at least one second block that have different glass transition temperatures (Tg), the said first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in documents EP 1 411 069 or WO 04/028 488.

The film-forming polymer may also be present in a composition of the invention in the form of particles dispersed in an aqueous phase or in a non-aqueous solvent phase, generally known as a latex or psuedolatex. Techniques for preparing these dispersions are well known to those skilled in the art.

Aqueous dispersions of film-forming polymers that may be used include the acrylic dispersions sold under the names Neocryl XK90®, Neocryl A1070®, Neocryl A1090®, Neocryl BT62®, Neocryl A1079® and Neocryl A523® by the company Avecia-Neoresins, Dow Latex 432® by the company Dow Chemical, Daitosol 5000 AD® or Daitosol 5000 SJ® by the company Daito Kasei Kogyo; Syntran 5760® by the company Interpolymer, Allianz OPT by the company Röhm & Haas, aqueous dispersions of acrylic or styrene/acrylic polymers sold under the brand name Joncryl® by the company Johnson Polymer, or the aqueous dispersions of polyurethane sold under the names Neorez R981® and Neorez R974® by the company Avecia-Neoresins, Avalure UR405®, Avalure UR410®, Avalure UR425®, Avalure UR450®, Sancure 875®, Sancure 861®, Sancure 878® and Sancure 2060® by the company Goodrich, Impranil 85® by the company Bayer and Aquamere H1511® by the company Hydromer; the sulfopolyesters sold under the brand name Eastman AQ® by the company Eastman Chemical Products, and vinyl dispersions, for instance Mexomer PAM® from the company Chimex, and mixtures thereof.

Examples of non-aqueous dispersions of film-forming polymers that may be mentioned include acrylic dispersions in isododecane, for instance Mexomer PAP® from the company Chimex, dispersions of particles of a grafted ethylenic polymer, which is preferably acrylic, in a liquid fatty phase, the ethylenic polymer advantageously being dispersed in the absence of additional surface stabilizer of particles, as described especially in document WO 04/055 081.

A composition according to the invention may also comprise a plasticizer that promotes the formation of a film with the film-forming polymer. Such a plasticizer may be chosen from any compound known to those skilled in the art as being capable of fulfilling the desired function.

Gelling Agents

A composition according to the invention may also comprise at least one hydrophilic or water-soluble gelling agent.

Hydrophilic or water-soluble gelling agents that may be mentioned include:

acrylic or methacrylic acid homopolymers or copolymers or the salts thereof and esters thereof and in particular the products sold under the names Versicol F or Versicol K by the company Allied Colloid, Ultrahold 8 by the company Ciba Geigy, and polyacrylic acids of Synthalen K type,

copolymers of acrylic acid and of acrylamide sold in the form of the sodium salt thereof under the names Reten by the company Hercules, the sodium polymethacrylate sold under the name Darvan No. 7 by the company Vanderbilt, and the sodium salts of polyhydroxycarboxylic acids sold under the name Hydagen F by the company Henkel,

polyacrylic acid/alkyl acrylate copolymers of Pemulen type,

AMPS (polyacrylamidomethylpropanesulfonic acid partially neutralized with aqueous ammonia and highly crosslinked) sold by the company Clariant,

AMPS/acrylamide copolymers of Sepigel or Simulgel type sold by the company SEPPIC, and

AMPS/polyoxyethylenated alkyl methacrylate copolymers (crosslinked or non-crosslinked), and mixtures thereof.

Other examples of water-soluble gelling polymers that may be mentioned include:

proteins, for instance proteins of plant origin such as wheat proteins and soybean proteins; proteins of animal origin such as keratins, for example keratin hydrolysates and sulfonic keratins;

anionic, cationic, amphoteric or nonionic chitin or chitosan polymers;

cellulose polymers such as hydroxyethylcellulose, hydroxypropylcellulose, methylcellulose, ethylhydroxyethylcellulose and carboxymethylcellulose, and also quaternized cellulose derivatives;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

associative polyurethanes such as the C16-OE120-C16 polymer from the company Servo Delden (sold under the name SER AD FX1100, this molecule bearing a urethane function and having a weight-average molecular weight of 1300), OE being an oxyethylene unit, Rheolate 205 bearing a urea function, sold by the company Rheox, or Rheolate 208 or 204 (these polymers being sold in pure form) or DW 1206B from Röhm & Haas bearing a C₂₀ alkyl chain and a urethane bond, sold at 20% solids in water. It is also possible to use solutions or dispersions of these associative polyurethanes, especially in water or in aqueous-alcoholic medium. Examples of such polymers that may be mentioned include SER AD FX1010, SER AD FX1035 and SER AD FX1070 from the company Servo Delden, and Rheolate 255, Rheolate 278 and Rheolate 244 sold by the company Rheox. It is also possible to use the products DW 1206F and DW 1206J, and also Acrysol RM 184 or Acrysol 44 from the company Röhm & Haas, or alternatively Borchigel LW 44 from the company Borchers,

optionally modified polymers of natural origin, such as:
gum arabic, guar gum, xanthan derivatives, karaya gum;
alginates and carrageenans;
glycoaminoglycans, hyaluronic acid and derivatives thereof;
shellac resin, sandarac gum, dammar resins, elemi gums and copal resins;
deoxyribonucleic acid;
mucopolysaccharides such as hyaluronic acid and chondroitin sulfates, and mixtures thereof.

Some of the water-soluble film-forming polymers mentioned above may also act as water-soluble gelling agent.

The hydrophilic gelling agents may be present in the compositions according to the invention in a content ranging from 0.05% to 40% by weight, preferably ranging from 0.1% to 20% by weight and preferentially ranging from 0.5% to 15% by weight, relative to the total weight of the composition.

Fillers

The composition according to the invention may also comprise at least one filler. These fillers serve especially to modify the rheology or the texture of the composition.

The fillers may be mineral or organic and of any shape, platelet-shaped, spherical or oblong, irrespective of the crystallographic form (for example lamellar, cubic, hexagonal, orthorhombic, etc.). Mention may be made of talc, mica, silica, silica surface-treated with a hydrophobic agent, fumed silica, kaolin, polyamide (Nylon®) powder (Orgasol® from Atochem), poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (Tospearls® from Toshiba, for example), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

It is also possible to use a compound that is capable of swelling on heating, and especially heat-expandable particles such as non-expanded microspheres of vinylidene chloride/acrylonitrile/methyl methacrylate copolymer or of acrylonitrile homopolymer or copolymer, for instance those sold, respectively, under the references Expancel® 820 DU 40 and Expancel® 007WU by the company Akzo Nobel.

The fillers may represent from 0.1% to 25% by weight and in particular from 0.2% to 20% by weight relative to the total weight of the composition.

Additional Fibres

The compositions in accordance with the invention may also comprise at least one additional fibre, other than the heat-retractable fibres used according to the invention, especially for obtaining an improvement in the lengthening effect of the mascara.

The additional fibres that may be used in the composition of the invention may be mineral or organic fibres, of synthetic or natural origin. They may be short or long, individual or organized, for example braided, and hollow or solid. They may have any shape and may especially have a circular or polygonal (square, hexagonal or octagonal) cross section depending on the specific application envisaged. In particular, their ends are blunted and/or polished to prevent injury.

In particular, the additional fibres have a length ranging from 1 μm to 10 mm, preferably from 0.1 mm to 5 mm and better still from 0.3 mm to 3 mm. Their cross section may be included in a circle with a diameter ranging from 2 nm to 500 μm, preferably ranging from 100 nm to 100 μm and better still from 1 μm to 50 μm. The weight or yarn count of fibres is often given in denier or decitex and represents the weight in grams per 9 km of yarn. Preferably, the fibres according to the invention have a yarn count chosen in the range from 0.01 to 10 denier, preferably from 0.1 to 2 denier and better still from 0.3 to 0.7 denier.

The additional fibres that may be used in the compositions according to the invention may be chosen from rigid or non-rigid fibres, and may be of synthetic or natural, mineral or organic origin.

Moreover, the additional fibres may or may not be surface-treated, may be coated or uncoated, and may be coloured or uncoloured.

As additional fibres that may be used in the compositions according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramid) fibres sold especially under the name Kevlar® by the company DuPont de Nemours.

The additional fibres may be present in a content ranging from 0.01% to 10% by weight, in particular from 0.1% to 5% by weight and more particularly from 0.3% to 3% by weight relative to the total weight of the composition.

The compositions according to the invention may also comprise any cosmetic active agent, such as active agents chosen from antioxidants, preserving agents, fragrances, bactericidal active agents or antiperspirants, neutralizers, emollients, moisturizers, thickeners, trace elements, softeners, sequestrants, acidifying or basifying agents, hydrophilic or lipophilic active agents, coalescers, plasticizers, vitamins, and screening agents, in particular sunscreens, and mixtures thereof.

Needless to say, a person skilled in the art will take care to select the optional additional compounds and/or the amount thereof such that the advantageous properties of the mascara composition used according to the invention are not, or are not substantially, adversely affected by the envisaged addition.

Physiologically Acceptable Medium

The compositions according to the invention may comprise a physiologically acceptable medium, i.e. a non-toxic medium that may be applied to human keratin materials and that has a pleasant appearance, odour and feel.

The physiologically acceptable medium is generally adapted to the nature of the support onto which the composition is to be applied, and also to the form in which the composition is to be conditioned.

The composition according to the invention may comprise an aqueous phase, which forms the continuous phase.

The term “composition with an aqueous continuous phase” means that the composition has a conductivity, measured at 25° C., of greater than or equal to 23 μS/cm (microSiemens/cm), the conductivity being measured, for example, using an MPC227 conductimeter from Mettler Toledo and an Inlab730 conductivity measuring cell. The measuring cell is immersed in the composition so as to remove the air bubbles that might be formed between the two electrodes of the cell. The conductivity reading is taken once the conductimeter value has stabilized. A mean is determined on at least three successive measurements.

The aqueous phase comprises water and/or at least one water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the compositions according to the invention may also be volatile.

Among the water-soluble solvents that may be used in the compositions in accordance with the invention, mention may be made especially of lower monoalcohols containing from 1 to 5 carbon atoms such as ethanol and isopropanol, glycols containing from 2 to 8 carbon atoms such as ethylene glycol, propylene glycol, 1,3-butylene glycol and dipropylene glycol, C₃-C₄ ketones and C₂-C₄ aldehydes.

The aqueous phase (water and optionally the water-miscible solvent) is generally present in the composition according to the present patent application in a content ranging from 1% to 80% by weight, preferably ranging from 10% to 70% by weight and preferentially ranging from 15% to 60% by weight relative to the total weight of the composition.

The composition according to the invention may be conditioned in a container delimiting at least one compartment that comprises the said composition, the said container being closed by a closing member.

The container may be in any suitable form. It may especially be in the form of a bottle, a tube, a jar or a case.

The closing member may be in the form of a removable stopper, a lid or a cover, especially of the type comprising a body fixed to the container and a cap articulated on the body. It may also be in the form of a member ensuring the selective closure of the container, especially a pump, a valve or a clapper.

The container may be combined with an applicator, especially in the form of a brush comprising an arrangement of bristles maintained by a twisted wire. Such a twisted brush is described especially in patent U.S. Pat. No. 4,887,622. It may also be in the form of a comb comprising a plurality of application members, obtained especially by moulding. Such combs are described, for example, in patent FR 2 796 529. The applicator may be in the form of a fine brush, as described, for example, in patent FR 2 722 380. The applicator may be in the form of a block of foam or of elastomer. The applicator may be free (sponge) or securely fastened to a rod borne by the closing member, as described, for example, in patent U.S. Pat. No. 5,492,426. The applicator may be securely fastened to the container, as described, for example, in patent FR 2 761 959.

The product may be contained directly in the container, or indirectly.

The closing member may be coupled to the container by screwing. Alternatively, the coupling between the closing member and the container is done other than by screwing, especially via a bayonet mechanism, by click-fastening or by gripping. The term “click-fastening” in particular means any system involving the crossing of a bead or cord of material by elastic deformation of a portion, especially of the closing member, followed by return to the elastically unconstrained position of the said portion after the crossing of the bead or cord.

The container may be at least partially made of thermoplastic material. Examples of thermoplastic materials that may be mentioned include polypropylene or polyethylene.

The container may have rigid or deformable walls, especially in the form of a tube or a tube bottle.

The container may comprise means for initiating or facilitating the distribution of the composition. By way of example, the container may have deformable walls so as to allow the composition to exit in response to a positive pressure inside the container, this positive pressure being caused by elastic (or non-elastic) squeezing of the walls of the container.

The container may be equipped with a drainer arranged in the region of the aperture of the container. Such a drainer makes it possible to wipe the applicator and possibly the rod to which it may be securely fastened. Such a drainer is described, for example, in patent FR 2 792 618.

According to one particular embodiment, a composition according to the invention may be a composition intended to be applied to the eyelashes, also known as a “mascara”. It may be a makeup composition, a base coat cosmetic composition, or a composition to be applied over a base coat cosmetic composition, also known as a top coat. The mascara is more particularly intended for human eyelashes, but also false eyelashes.

The compositions used according to the invention may be manufactured via the known processes, generally used in the field of cosmetics.

The heating performed after the step of applying the mascara composition may be performed via any suitable means for heating keratin materials to a temperature ranging from 45 to 100° C., preferably between 50 and 70° C. and preferably between 55 and 65° C. Among these heating means are heating combs and heating tongs.

The invention is illustrated in greater detail in the examples that follow, which are given as non-limiting illustrations of the invention.

### EXAMPLES

In the following compositions, the ingredients are given in grams of active material.

### Example 1

Heat-retractable fibres are prepared in the following manner. A PETG 5116 filament (available from Eastman Chemical Products, Inc., Kingsport, Tenn.) is extruded so as to have a diameter of 20 microns. The filament thus obtained is then drawn to a ratio of 1/1.5 in a bath of water heated to 70° C. according to the standard methods known to those skilled in the art. The fibres are then chopped again so as not to exceed a length of 3 mm.

The fibres thus obtained are used to make the following composition:

| | |
|---|---|
| Baycusan C1001 (available from Bayer) | 45% |
| Hydroxyethylcellulose (Cellosize QP4400 H - | 1.7% |
| Amerchol) | |
| Fumed silica (Aerosil 200 - Degussa) | 0.9% |
| Pigments | 5% |
| Heat-retractable fibres | 10% |
| Water | 37.4% |

The composition obtained is applied to the eyelashes using a mascara brush. After drying, a heating probe at 70° C. of cylindrical shape 3 mm in diameter is used, which is applied onto the dry deposit so as to activate the retraction of the fibres. An increase in the curling of the eyelashes is then observed.

### Example 2

Heat-retractable fibres are prepared according to the protocol described in Example 3 of patent application U.S. Pat. No. 5,439,438 (before the manufacture of the nonwoven by embossing). The fibres are then chopped again so as not to exceed a length of 3 mm. The fibres thus obtained are used to make the following composition:

| | |
|---|---|
| Baycusan C1001 (available from Bayer) | 45% |
| Hydroxyethylcellulose (Cellosize QP4400 H - | 1.7% |
| Amerchol) | |
| Fumed silica (Aerosil 200 - Degussa) | 0.9% |
| Pigments | 5% |
| Heat-retractable fibres | 10% |
| Water | 37.4% |

The composition obtained is applied to the eyelashes using a mascara brush. After drying, a heating probe at 70° C. of cylindrical shape 3 mm in diameter is used, which is applied onto the dry deposit so as to activate the retraction of the fibres. An increase in the curling of the eyelashes is then observed.

1. Composition for making up and/or caring for keratin fibres, comprising a physiologically acceptable medium and characterized in that it contains fibres that are capable of changing in length and/or shape in response to an external stimulus, the said fibres being made of a material chosen from polyethylene terephthalate and derivatives thereof.

2. Composition according to claim 1, in which the fibres are fibres that are retractable at a temperature T₁, which is less than or equal to 80° C.

3. Composition according to claim 1, in which the said fibres are made of a material chosen from polyethylene terephthalate copolymers, polyethylene terephthalate glycol copolymers, and mixtures of polybutylene terephthalate and polyethylene terephthalate.

4. Process for making up and/or caring for keratin fibres, comprising at least the following steps, in this order:
applying the composition defined in claim 1,
heating the keratin fibres to a temperature ranging from T₁ to 100° C.

5. Process for making up and/or caring for keratin fibres according to claim 4, in which the length of the retractable fibres ranges from 0.1 mm to 10 mm, preferably from 0.5 mm to 5 mm and better still from 1 mm to 3 mm.

6. Process for making up and/or caring for keratin fibres according to claim 4, in which the diameter of the retractable fibres ranges from 5 to 50 μm and preferably from 10 to 50 μm.

7. Process for making up and/or caring for keratin fibres according to claim 4, in which the amount of retractable fibres ranges from 5% to 50% and preferably from 10% to 20% by weight relative to the total weight of the composition.

8. Process for making up and/or caring for keratin fibres according to claim 4, in which the composition comprises at least one wax with a melting point of greater than 80° C.

9. Process for making up and/or caring for keratin fibres according to claim 8, in which the wax is a wax with a hardness of greater than 6 MPa.

10. Process for making up and/or caring for keratin fibres according to claim 9, in which the hard wax is chosen from carnauba wax, candelilla wax and polyethylene waxes.

11. Process for making up and/or caring for keratin fibres according to claim 8, in which the amount of wax ranges from 0.1% to 50% by weight relative to the total weight of the composition.

12. Process for making up and/or caring for keratin fibres according to claim 4, in which the composition is free of wax.

13. Process for making up and/or caring for keratin fibres according to claim 4, characterized in that the composition comprises at least one ingredient chosen from pasty compounds, film-forming polymers, gelling agents, fillers and additional fibres.

14. Process for making up and/or caring for keratin fibres according to claim 4, in which the composition comprises an active agent chosen from antioxidants, preserving agents, fragrances, bactericidal active agents or antiperspirants, neutralizers, emollients, moisturizers, thickeners, trace elements, softeners, sequestrants, acidifying or basifying agents, hydrophilic or lipophilic active agents, coalescers, plasticizers, vitamins, and screening agents, in particular sunscreens, and mixtures thereof.

15. Use in a mascara composition of fibres that are capable of changing in length and/or shape when they are subjected to a temperature of less than or equal to 80° C., the said fibres being made of a material chosen from polyethylene terephthalate and derivatives thereof, for increasing the level of curling of the eyelashes and/or for increasing the uniformity of curling of the eyelashes.
